(19) United States
(12) Patent Application Publication
(10) Pub. No.: US 2021/0282857 A1
(43) Pub. Date: Sep. 16, 2021

(54) INTERNAL LASER LIGHT DELIVERY FOR PHOTOACOUSTIC-GUIDED DRILLING

(71) Applicant: The Johns Hopkins University, Baltimore, MD (US)

(72) Inventors: Muyinatu BELL, Baltimore, MD (US); Joshua SHUBERT, Santa Monica, CA (US)

(73) Assignee: The Johns Hopkins University, Baltimore, MD (US)

(21) Appl. No.: 17/260,943

(22) PCT Filed: Jul. 15, 2019
(86) PCT No.: PCT/US2019/041839

§ 371 Date: Jan. 15, 2021

Related U.S. Application Data
(60) Provisional application No. 62/699,483, filed on Jul. 17, 2018.

(51) Int. Cl. A61B 18/26; A61B 17/16

(52) U.S. Cl. CPC A61B 18/26; A61B 2018/00339; A61B 17/1626; A61B 17/1615

(57) ABSTRACT

A may comprise a first optical fiber and an optical interface connected to the first optical fiber. The optical interface may be configured to transfer laser light from the first optical fiber to a second optical fiber in a drill bit. The second optical fiber may propagate the laser light to a face of the drill bit to create an ultrasonic emission.

### RELATED APPLICATION

This application claims priority under 35 U.S.C. § 119 to U.S. Provisional Patent Application No. 62/699,483, filed on Jul. 17, 2018, and entitled “INFERNAL LIGHT DELIVERY FOR PHOTOACOUSTIC-GUIDED DRILLING,” the content of which is incorporated by reference herein in its entirety.

### GOVERNMENT LICENSE RIGHTS

This invention was made with U.S. Government support under grant EB018994 awarded by the National Institutes of Health. The government has certain rights in the invention.

### BACKGROUND

Photoacoustic imaging delivers laser light to biological tissue, where energy from the laser light is absorbed by the biological tissue and converted to heat, which produces an ultrasonic emission comprising one or more ultrasonic waves. The ultrasonic emission can be captured by an ultrasound transducer and analyzed to determine a composition of the biological tissue (e.g., bone, nerve, blood vessel, and/or the like).

### SUMMARY

According to some implementations, a device may include a first optical fiber and an optical interface connected to the first optical fiber, wherein the optical interface is configured to transfer laser light from the first optical fiber to a second optical fiber in a drill bit.

According to some implementations, a shaft may include a face of the shaft that is configured to penetrate bone and an optical fiber within the shaft to propagate laser light generated by a laser light source to the face of the shaft to create an ultrasonic emission at the face of the shaft.

According to some implementations, a method may include causing, by a device, a light source to generate laser light, wherein the laser light propagates through an optical fiber of a drill bit to create an ultrasonic emission at a face of the drill bit; obtaining, by the device and from an ultrasound transducer device, ultrasound data associated with the ultrasonic emission; processing, by the device, the ultrasound data to determine a location of the drill bit tip; processing, by the device, the ultrasound data to determine a type of tissue at the face of the drill bit; and causing, by the device, at least one action to be performed based on determining the type of tissue at the face of the drill bit.

### BRIEF DESCRIPTION OF THE DRAWINGS

FIG. 1 is a diagram of an example implementation described herein.

FIG. 2 is a diagram of an example optical interface described herein.

FIG. 3 is a diagram of example drill bits described herein.

FIGS. 4A-4C are diagrams of example implementations described herein

FIG. 5 is a diagram of an example surgical procedure using the devices described herein.

FIG. 6 is a diagram of an example environment in which systems and/or methods described herein may be implemented.

FIG. 7 is a diagram of example components of one or more devices of FIG. 6.

FIG. 8 is a flowchart of an example process for internal laser light delivery for photoacoustic-guided drilling.

### DETAILED DESCRIPTION

The following detailed description of example implementations refers to the accompanying drawings. The same reference numbers in different drawings may identify the same or similar elements.

In some cases, a surgeon may perform a spinal fusion surgery to repair damaged vertebrae within a patient's spinal column During the spinal fusion surgery, the surgeon may affix an implant (e.g., a screw) to a pedicle of a damaged vertebra. A pedicle has cylindrical-like geometry and runs along either side of a vertebra, connecting the vertebral body to the spinal column. The pedicle includes a cortical bone outer region that surrounds a cancellous bone core. The surgeon may affix the implant to the pedicle by using a surgical drill to drill a hole in the pedicle to create a passage in the cancellous bone core of the pedicle. The surgeon then may insert the implant in the passage.

In some cases, the surgeon may drill a hole that breaches the cortical bone outer region of the pedicle. This may affect the structural integrity of the pedicle and may cause damage to adjacent critical structures, such as the spinal cord, nerves, blood vessels, and/or the like. In many cases, different types of imaging technology have been used to assist the surgeon in drilling the pedicle and minimizing the potential for breaching the cortical bone outer region of the pedicle and causing damage to the critical structures. For example, computed tomography (CT) scan images, fluoroscopy images, ultrasound images, and/or computed axial tomography (CAT) scan images of the pedicle may be taken prior to surgery to estimate a diameter and a length of the pedicle, as well as an entry point and a trajectory for the implant. However, the different types of imaging technology cannot be used in real-time, or in the case of fluoroscopy, may expose the surgeon and the patient to potentially large amounts of ionizing radiation. Further, the different types of imaging technology are bulky, cumbersome, and require a trained technician to operate.

In some cases, a surgical tool, such as a surgical drill, may be augmented with an external laser light delivery system to utilize photoacoustic imaging at a tip of the surgical tool, such as at a drill tip location of the surgical drill. However, an external light delivery system requires a straight “line of sight” line between optical fibers transmitting the laser light and the surgical tool tip. An external light delivery system that is mounted on the surgical tool also increases a form factor size of the surgical tool, rendering the surgical tool unusable when drilling holes deeper than a few millimeters (e.g., because the laser light will be absorbed by surrounding tissue in a patient before it can reach the tool tip). The external light delivery system may also be problematic when the external light delivery system prohibits the surgical tool from reaching a surgical target within the patient.

Some implementations described herein provide a surgical device that includes an optical fiber and an optical interface within the surgical device. In some implementations, a laser light source may generate laser light that propagates through the optical fiber to the optical interface. In some implementations, the optical interface may be configured to transfer the laser light from the optical fiber to an internal optical fiber in a shaft instrument (e.g., a drill bit, a needle, a probe, and/or the like that can be inserted into and/or attached to the surgical device). In some implementations, the laser light may propagate through the internal optical fiber to create an ultrasonic emission (also referred to as a photoacoustic signal) at a face of the shaft instrument. In some implementations, an ultrasound transducer device may obtain ultrasound data associated with the ultrasonic emission, and a processing device may process the ultrasound data to determine a type of biological tissue at the face of the shaft instrument. In some implementations, the processing device may cause one or more actions to be performed based on determining the type of biological tissue, such as cause information concerning the type of biological tissue to be displayed on a display screen.

In this way, a surgeon may receive real-time information concerning what type of biological tissue is at the face of the shaft instrument, and the surgeon may adjust operation of the surgical device accordingly. For example, the surgeon may determine a starting point and/or a drilling path for drilling a pedicle based on identifying cancellous or cortical bone of the pedicle at a face of a drill bit of the surgical device. This can reduce a likelihood that the surgeon may drill a hole that breaches the cortical bone outer region of the pedicle.

Further, because laser light is propagated internally in the surgical device, the form factor of the surgical device can be smaller and therefore easier to operate than a similar device that uses an external laser light delivery system. Moreover, because the laser light is propagated to the face of the shaft instrument, some implementations enable photoacoustic imaging to be used even when drilling deep holes (e.g., a hole with a greater depth than a few millimeters). Additionally, some implementations provide a safer working environment for the surgeon and/or the patient (e.g., because photoacoustic imaging does not expose the surgeon to harmful ionizing radiation).

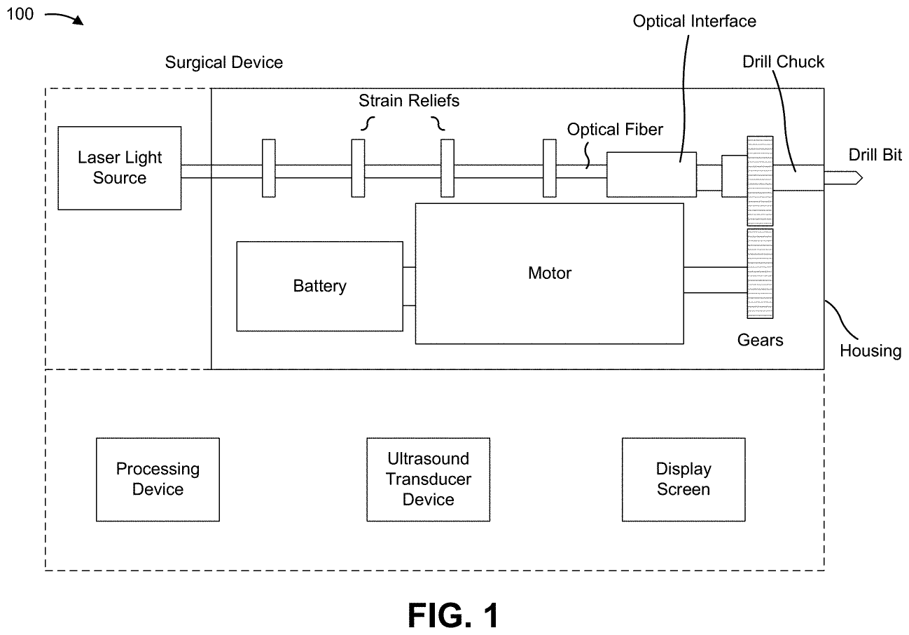

FIG. 1 is a diagram of an example implementation 100 described herein. As shown in FIG. 1, example implementation 100 may include a surgical device. The surgical device may be a surgical drill device, a neurosurgical drill device (e.g., that has a smaller form factor than a surgical drill device), and/or the like. The surgical device may comprise a battery (e.g., a 9 volt battery, a lithium-ion battery, and/or the like), a motor, an optical fiber, an optical interface (also referred to, in some implementations, as an optical fiber drill bit interface (OFDBI)), one or more gears, a drill bit, a structure to hold the drill bit (e.g., a drill chuck), an operating switch (not shown), and/or the like. In some implementations, the battery may provide electrical energy to the motor (e.g., when the operating switch is engaged), which may convert the electrical energy into mechanical energy to turn the one or more gears. In some implementations, the one or more gears may be connected to the drill bit (e.g., via the structure to hold the drill bit) and may cause the drill bit to rotate (e.g., spin clockwise or counterclockwise on an axis). In some implementations, the drill bit may include an internal optical fiber (e.g., within a shaft of the drill bit).

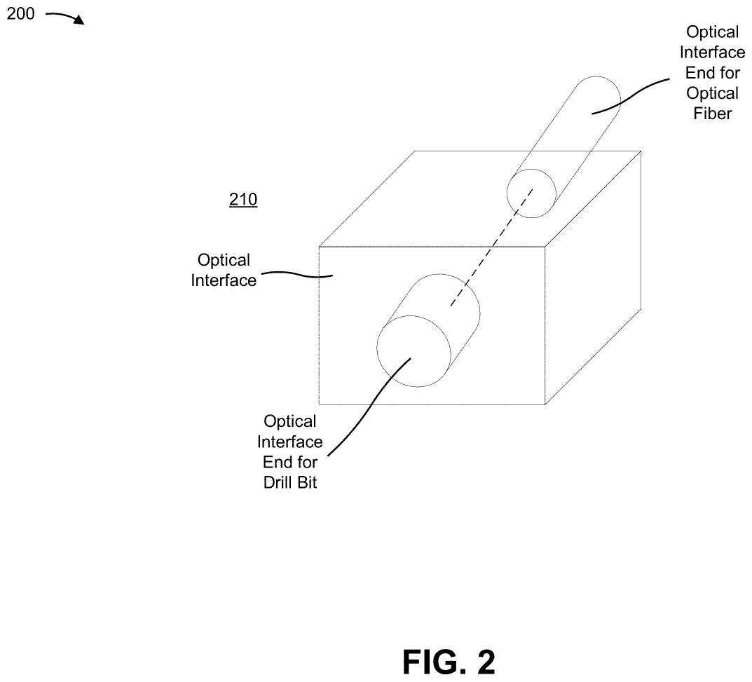

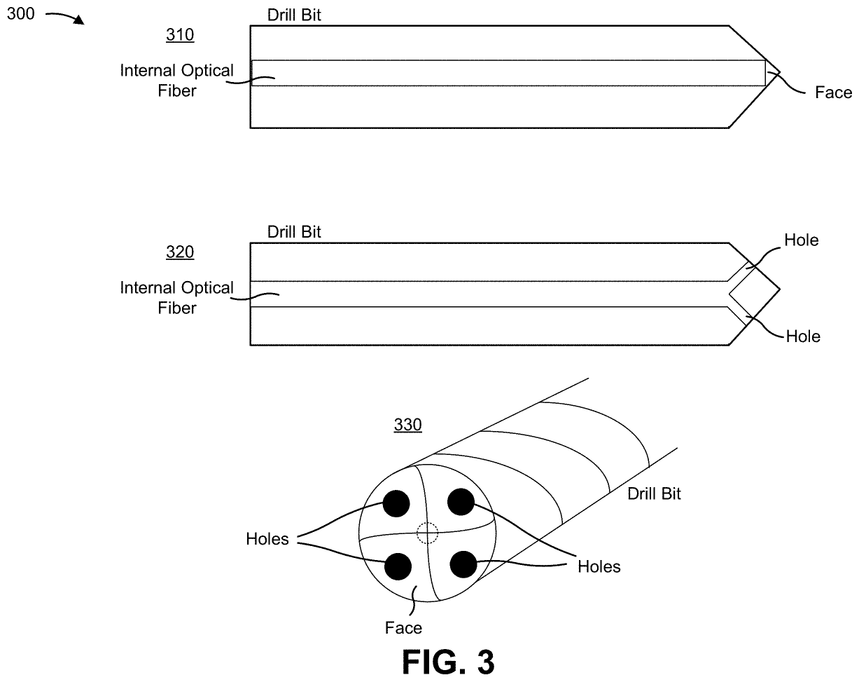

As shown in FIG. 1, example implementation 100 may also include a laser light source (e.g., a laser diode, such as a pulsed laser diode). In some implementations, the surgical device may include the laser light source (e.g., the laser light source may be within a housing of the surgical device) and/or the laser light source may be external to the surgical device (e.g., the laser light source may be outside the housing of the surgical device). The laser light source may be connected to the optical fiber. In some implementations, the laser light source may generate laser light and the laser light may travel to the optical fiber (e.g., directly from the laser light source, via one or more additional structures (e.g., an additional optical fiber), and/or the like). The optical fiber may propagate the laser light (e.g., within the surgical device) to the optical interface. The optical interface may be connected to the optical fiber and the drill bit, and may transfer the laser light (e.g., via an open-air chamber) from the optical fiber to the internal optical fiber of the drill bit. The laser light may propagate in the internal optical fiber of the drill bit to a face of the drill bit, which may cause a biological tissue (e.g., a bone, a blood vessel, a nerve, and/or the like) at a face of the drill bit to absorb the laser light and create an ultrasonic emission composed of one or more ultrasonic waves. This is sometimes referred to as creating a photoacoustic signal at the face of the drill bit. Further details regarding the optical interface and the drill bit are provided herein in relation to FIGS. 2 and 3, respectively.

As shown in FIG. 1, example implementation 100 may also include a processing device, an ultrasound transducer device, a display screen and/or the like. In some implementations, the surgical device may include and/or be connected to the processing device, the ultrasound transducer device, and/or the display screen. The ultrasound transducer device may obtain ultrasound data associated with the ultrasonic emission. The processing device may be connected to the ultrasound transducer device and may process and/or analyze the ultrasound data. The processing device may be connected to the display screen and may cause information concerning the ultrasound data to be displayed on the display screen.

In some implementations, the surgical device, the laser light source, the processing device, the ultrasound transducer device, the display screen, and/or the like may communicate with each other using a wired connection, a wireless connection, or a combination of wired and wireless connections. For example, the surgical device, the laser light source, the processing device, the ultrasound transducer device, and/or the display screen may communicate via a non-cellular, wireless connection, such as a Wi-Fi connection, a Bluetooth connection (e.g., a Bluetooth connection that supports mesh networking), a Zigbee connection (e.g., a Zigbee connection that supports mesh networking), a near field communication (NFC) connection, and/or the like. As another example, the surgical device, the laser light source, the processing device, the ultrasound transducer device, and/or the display screen may communicate via a wired connection, such as a universal serial bus (USB) connection, an Ethernet connection, and/or the like.

As shown in FIG. 1, the surgical device may include a housing to structurally support and/or encapsulate the battery, the motor, the optical fiber, the optical interface, the one or more gears, the laser light source, the processing device, the ultrasound transducer device, the display screen, and/or the like within the housing. In some implementations, the surgical device may include attachment points (e.g., shown in FIG. 1 as strain reliefs) that physically secure the optical fiber (e.g., without bending the optical fiber), the laser light source, the optical interface, and/or the like to the housing. This ensures that the optical fiber, the laser light source, the optical interface, and/or the like remain stationary relative to motion of the motor, the one or more gears, the drill bit, and/or the like, and/or that the optical fiber, the laser light source, the optical interface are not accidentally damaged by an operator's handling of the surgical device.

As indicated above, FIG. 1 is provided merely as an example. Other examples may differ from what is described with regard to FIG. 1. Further, the number and arrangement of devices shown in FIG. 1 are provided as an example. In practice, there may be additional devices, fewer devices, different devices, or differently arranged devices than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g., one or more devices) of example implementation 100 may perform one or more functions described as being performed by another set of devices of example implementation 100.

FIG. 2 is a diagram of an example optical interface described herein. As shown by reference number 210, the optical interface may include a first end for an optical fiber (e.g., an optical fiber that propagates laser light from the laser light source to the optical interface, as described herein) and a second end for a drill bit (e.g., a drill bit with an internal optical fiber, as described herein). The optical interface may connect to the optical fiber (e.g., an end of the optical fiber may be inserted into the first end of the optical interface) and/or may connect to the drill bit (e.g., a shank of the drill bit may be inserted into the second end of the optical interface). The first end of the optical interface may be configured to stabilize the optical fiber so that it does not move. The second end of the optical interface may be configured to allow the drill bit to rotate in place (e.g., spin about an axis). The optical interface may position the optical fiber and the drill bit (e.g., align the end of the optical fiber and the shank of the drill bit) to allow laser light to transfer from the optical fiber to the internal optical fiber of the drill bit. That is, the optical interface may position the optical fiber and the drill bit to allow laser light that propagated through the optical fiber to exit the optical fiber at the end of the optical fiber, to travel an open-air distance inside the optical interface (e.g., inside a chamber of the optical interface), and to enter the internal optical fiber of the drill bit.

As shown in FIG. 2 (e.g., by the dotted line), the first end for the optical fiber and the second end for the drill bit of the optical interface may be separated by a distance to allow the shank of the drill bit to avoid touching and thereby damaging the end of the optical fiber when the drill bit rotates (e.g., because of friction, heat, and/or the like). In some implementations, the distance may be optimized to minimize a likelihood of the shank of the drill bit touching the end of the optical fiber and to maximize an amount of laser light that is transferred from the optical fiber to the internal optical fiber of the drill bit. Accordingly, the optical interface may be configured such that the distance satisfies a threshold (e.g., the distance is greater than a minimal optimal distance, such as 1 mm, and/or less than or equal to a maximum optimal distance, such as 5 mm).

As indicated above, FIG. 2 is provided merely as an example. Other examples may differ from what is described with regard to FIG. 2.

FIG. 3 depicts diagrams of example drill bits described herein. As shown by reference numbers 310 and 320, a drill bit may have an internal optical fiber to propagate laser light to a face of the drill bit. The internal optical fiber may comprise one or more polished glass fibers, one or more plastic fibers, and/or the like. The internal optical fiber may be in a hollow core of the drill bit (e.g., inserted into the hollow core, chemically grown in the hollow core, and/or the like). The face of the drill bit may be configured to penetrate bone and/or similar tissue (e.g., the face of the drill bit may include a tip and/or one or more cutting edges to cut away bone to create a hole in the bone). In some implementations, the drill bit may be composed of resin, steel, and/or a material that absorbs laser light. The drill bit may accommodate different form factors of the surgical device, such as various drill diameters, various spiral patterns, various point angles, various lips, various lengths, and/or the like associated with the surgical device and/or the structure to hold the drill bit.

In some implementations, the drill bit may have a length (e.g., a distance from an end of the shank of the drill bit to the face of the drill bit) and/or a width (e.g., a diameter of the shaft of the drill bit). Similarly, the internal optical fiber may have a length (e.g., a distance from one end of the internal optical fiber to another end of the internal optical fiber) and/or a width (e.g., a diameter of the internal optical fiber). The internal optical fiber may have a length that is less than the length of the drill bit (e.g., the internal optical fiber may have a length of 62 mm when the length of the drill bit is 65 mm) and/or may have a width that is less than the width of the drill bit (e.g., the internal optical fiber may have a width of 1 mm when the width of the drill bit is 5 mm).

As shown by reference number 310, the drill bit may have a solid face (e.g., the internal optical fiber may be contained within the drill bit without direct access to the face of the drill bit). Laser light may propagate through the internal optical fiber of the drill bit (e.g., after the light laser was transferred to the internal optical fiber of the drill bit via the optical interface, as described herein) and may exit the internal optical fiber at the face of the drill bit (e.g., due to the proximity of the end of the internal optical fiber to the face of the drill bit) to interact with tissue at the face of the drill bit (e.g., to stimulate the tissue to create an ultrasonic emission).

As shown by reference numbers 320 and 330, the drill bit may have one or more holes in the face of the drill bit (e.g., the one or more holes may provide the internal optical fiber access to the face of the drill bit). In some implementations, the one or more holes may be filled with a material that propagates laser light and/or protects the internal optical fiber from being damaged or blocked by operation of the drill bit (e.g., to prevent the one or more holes from clogging with tissue components, such as bone fragments, blood, and/or the like). For example, the one or more holes may be implanted with diamond lenses (e.g., artificial diamond lenses, such as artificial diamond lenses created by a chemical deposition process). Laser light may propagate through the internal optical fiber of the drill bit (e.g., after the light laser is transferred to the internal optical fiber of the drill via the optical interface, as described herein) and may exit the internal optical fiber at the face of the drill bit via the one or more holes to interact with tissue at the face of the drill bit (e.g., to stimulate the tissue at one or more points to create one or more ultrasonic emissions in a region in front of the face of the drill bit).

While some implementations described herein concern a drill bit, additional instruments with an internal optical fiber are also contemplated. For example, implementations include any shaft instrument with a face that is configured to penetrate bone and/or similar tissue, such as a drill bit, a needle, a probe (e.g., a pedicle probe), and/or the like. Each shaft instrument may have a respective internal optical fiber. For each shaft instrument, the internal optical fiber may propagate laser light to the face of the shaft instrument in a similar manner as described herein. Moreover, the face of the shaft instrument may include one or more holes to facilitate the laser light exiting the internal optical fiber at the face of the shaft instrument to interact with tissue at the face of the shaft instrument, in a similar manner as described herein.

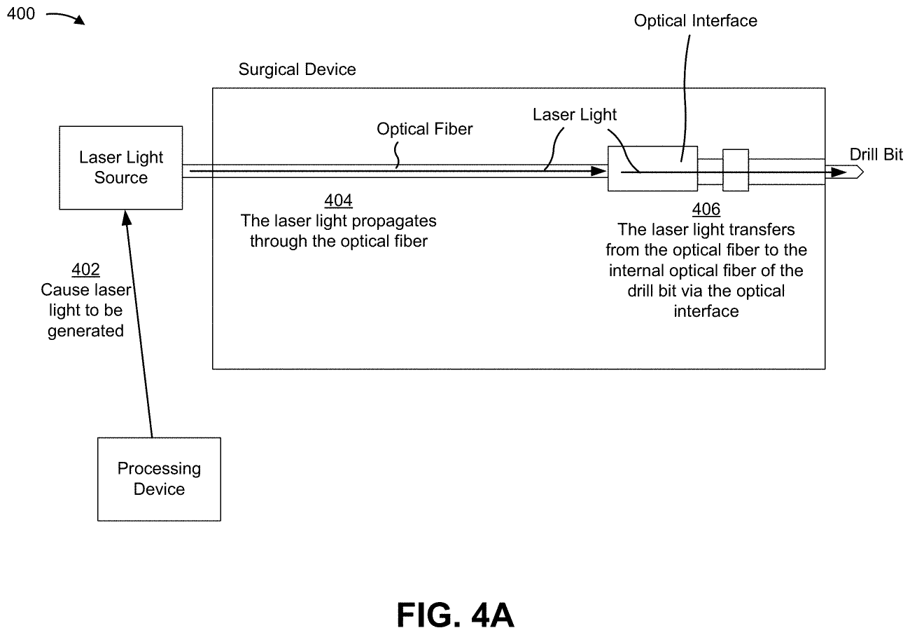

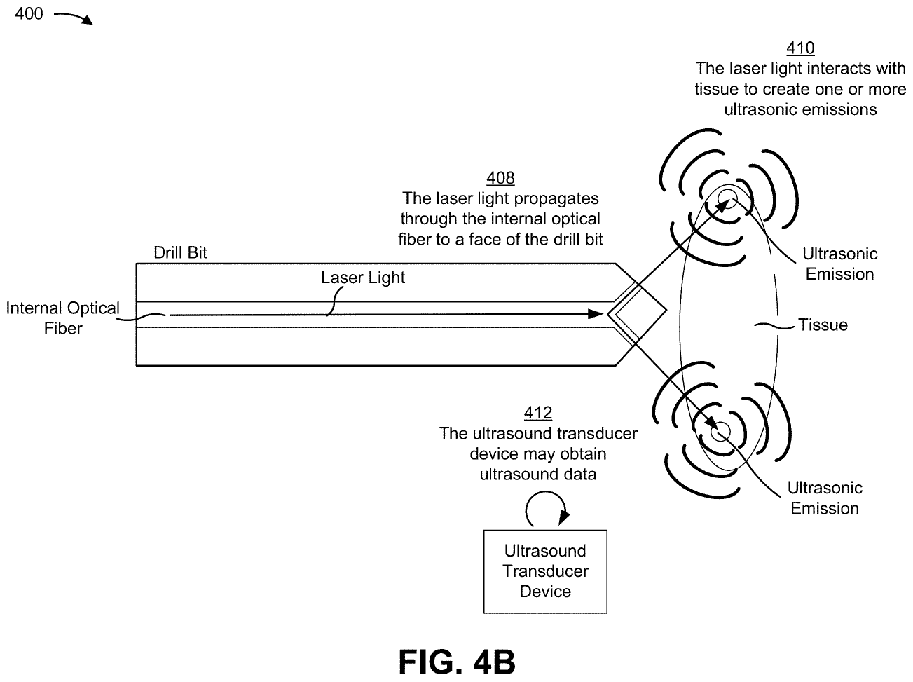

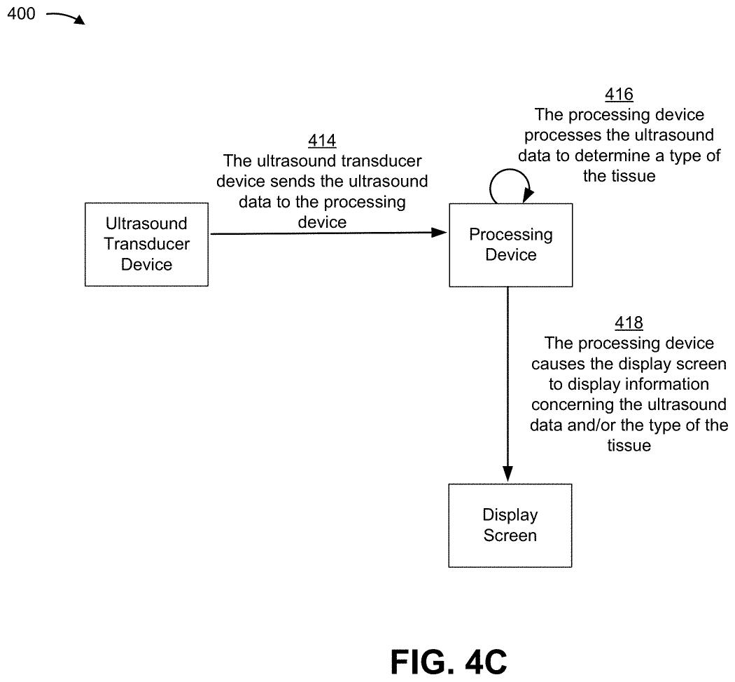

FIGS. 4A-4C are diagrams of one or more example implementations 400 described herein. As shown in FIG. 4A and by reference number 402, the processing device may cause the laser light source to generate laser light. In some implementations, a user (e.g., an operator of the surgical device and/or the processing device) may interact with a user interface of the processing device to cause the processing device to run a program (e.g., a mobile device application, a desktop computer application, a cloud-based application, and/or the like) that causes the laser light source to generate the laser light. The user may interact with the user interface to specify a preference concerning an energy, a fluence, a wavelength, and/or the like of laser light to be generated by the laser light source. Accordingly, the processing device may send a signal to the laser light source that causes the laser light source to activate, turn on, generate laser light according to the preference of the user, and/or the like. Additionally, or alternatively, the user may manually activate, turn on, and/or the like the laser light source and may manually input parameters concerning an energy, a fluence, a wavelength, and/or the like of laser light to cause the laser light source to generate the laser light according to the user preference.

In some implementations, the user preference may be associated with a particular tissue type. For example, the user may specify an energy, a fluence, a wavelength, and/or the like associated with detecting blood vessels. The laser light source may then generate laser light that is well absorbed by blood vessels to create an ultrasonic emission that can be detected by the ultrasound transducer device and/or identified by the processing device. Similarly, the user preference may be associated with one or more other tissue types such as bones (e.g., cancellous bone, cortical bone, and/or the like), nerves, and/or the like and the laser light source may generate laser light that is well absorbed by one or more of the other tissue types. In some implementations, the laser light source may generate laser light within a wavelength range (e.g., greater than 600 nm and less than or equal to 2400 nm), within an energy range (e.g., greater than 0.12 mJ and less than or equal to 2.72 mJ), within a fluence range (e.g., greater than 15 mJ/cm² and less than or equal to 345 mJ/cm²), and/or the like.

As shown by reference number 404, the laser light may propagate from the laser light source through the optical fiber of the surgical device to the optical interface. The optical fiber may include fibers comprised of glass, plastic, and/or the like to mitigate loss of the laser light as the laser light propagates through the optical fiber. In some implementations, the optical fiber may have a 1 mm diameter and/or may have a diameter within a diameter range (e.g., greater than 0.5 mm and less than or equal to 5 mm). As shown by reference number 406, the laser light may transfer from the optical fiber to the internal optical fiber of the drill bit via the optical interface in a similar manner as described herein in relation to FIG. 2.

As shown by FIG. 4B and by reference number 408, the laser light may propagate through the internal optical fiber to a face of the drill bit (e.g., via one or more holes in the face of the drill bit). As shown by reference number 410, the laser light may interact with tissue at the face of the drill bit (e.g., by stimulating the tissue) to create one or more ultrasonic emissions (also referred to as photoacoustic signals).

As shown by reference number 412, the ultrasound transducer device may obtain ultrasound data associated with the one or more ultrasonic emissions. For example, a user of the ultrasound transducer device (e.g., the operator of the surgical device) may position the ultrasound transducer device at or near the face of the drill bit (e.g., at a position on the tissue) to detect the one or more ultrasonic emissions. In some implementations, the ultrasound transducer device may be a linear ultrasound transducer array with a bandwidth range greater than 3 MHz and less than or equal to 8 MHz. Additionally, or alternatively, the ultrasound transducer device may be a phase ultrasound transducer array with a bandwidth range greater than 1 MHz and less than or equal to 5 MHz.

As shown in FIG. 4C and by reference number 414, the processing device may obtain the ultrasound data from the ultrasound transducer device. For example, the ultrasound transducer device may send the ultrasound data to the processing device, and/or the processing device may request and receive the ultrasound data. As shown by reference number 416, the processing device may process the ultrasound data. In some implementations, the processing device may analyze the ultrasound data to determine a type of the tissue at the face of the drill bit. For example, the processing device may analyze the ultrasound data to determine whether the tissue is bone, a blood vessel, a nerve, and/or the like. As another example, when the tissue is bone, the processing device may determine whether the tissue is cortical bone, cancellous bone, and/or the like.

In some implementations, the processing device may process the ultrasound data to determine a contrast and signal-to-noise ratio (SNR) associated with the ultrasound data to facilitate determining the type of tissue at the face of the drill bit. For example, the processing device may apply the following formulas to the ultrasound data to determine the contrast and the SNR:

$$Contrast = 20 log ( \frac{{μ}_{signal}}{{μ}_{background}} )$$

$$SNR = \frac{{μ}_{signal}}{{σ}_{signal}}$$

where μ_{signal} and σ_{signal} are the mean and standard deviation of ultrasonic signals within a region of interest of an image associated with the ultrasound data, and μ_{background} is the mean of ultrasonic signals within a region of interest in a background of the image.

In some implementations, the processing device may process the ultrasound data using a machine learning model to determine the type of tissue at the face of the drill bit. In some implementations, the processing device, or a different device (such as a server device), may generate and/or train the machine learning model to determine the type of tissue at the face of the drill bit. For example, the processing device may obtain and process historical information (e.g., historical information concerning ultrasound data associated with stimulating different types of biological tissue with laser light, information concerning analysis of the ultrasound data (e.g. information concerning determinations on whether particular ultrasound data is associated with particular types of tissue), and/or the like to generate and/or train the machine learning model to determine a type of tissue at the face of the drill bit based on ultrasound data.

In some implementations, to generate the machine learning model, the processing device may perform a set of data manipulation procedures to pre-process the historical information. The monitoring platform may use a data pre-processing procedure, a model training procedure, a model verification procedure, and/or the like to pre-process the historical information to generate processed historical information. For example, the monitoring platform may pre-process the historical information to remove irrelevant information, confidential data, corrupt data, and/or the like; to replace personal information with generic information; to infer and/or to address missing information and/or to remove records that include missing information; and/or the like. In this way, the monitoring platform may organize thousands, millions, or billions of data entries for machine learning and model generation.

In some implementations, the processing device may perform a training operation when generating the machine learning model. For example, the monitoring platform may portion the historical information into a training set (e.g., a set of data to train the model), a validation set (e.g., a set of data used to evaluate a fit of the model and/or to fine tune the model), a test set (e.g., a set of data used to evaluate a final fit of the model), and/or the like. In some implementations, a minimum feature set may be created from pre-processing and/or dimensionality reduction of the historical information. In some implementations, the processing device may train the machine learning model on this minimum feature set, thereby reducing processing required to train the machine learning model, and may apply a classification technique to the minimum feature set.

When training the machine learning model, the processing device may utilize a random forest classifier technique to train the machine learning model. For example, the processing device may utilize a random forest classifier technique to construct multiple decision trees during training and may output a classification of the historical information. As another example, the processing device may utilize a random forest regression technique to construct multiple decision trees during training and may output a numeric predication associated with the historical information. Additionally, or alternatively, when training the machine learning model, the processing device may utilize one or more gradient boosting techniques, such as an xgboost classifier technique, an xgboost regression technique, a gradient boosting machine (GBM) technique, a gradient boosting tree, and/or the like to generate a prediction model from a set of weak prediction models.

When training the machine learning model, the processing device may utilize a logistic regression technique to train the machine learning model. For example, the processing device may utilize a binary classification of the historical information (e.g., whether the historical information is indicative of a particular accurate prediction), a multi-class classification of the historical information (e.g., whether the historical information is indicative of one or more accurate predictions), and/or the like to train the machine learning model. Additionally, or alternatively, when training the machine learning model, the processing device may utilize a naïve Bayes classifier technique to train the machine learning model. For example, the behavioral analytics platform may utilize binary recursive partitioning to divide the historical information into various binary categories (e.g., starting with whether the historical information is indicative of a particular accurate prediction). Based on using recursive partitioning, the processing device may reduce utilization of computing resources relative to manual, linear sorting and analysis of data points, thereby enabling use of thousands, millions, or billions of data points to train a machine learning model, which may result in a more accurate machine learning model than using fewer data points.

Additionally, or alternatively, when training the machine learning model, the processing device may utilize a support vector machine (SVM) classifier technique. For example, the processing device may utilize a linear model to implement non-linear class boundaries, such as via a max margin hyperplane. Additionally, or alternatively, when utilizing the SVM classifier technique, the processing device may utilize a binary classifier to perform a multi-class classification. Use of an SVM classifier technique may reduce or eliminate overfitting, may increase a robustness of the machine learning model to noise, and/or the like.

In some implementations, the processing device may train the machine learning model using a supervised training procedure. In some implementations, the processing device may receive additional input to the machine learning model from a subject matter expert. In some implementations, the processing device may use one or more other model training techniques, such as a neural network technique, a latent semantic indexing technique, and/or the like. For example, the processing device may perform a multi-layer artificial neural network processing technique (e. g, using a recurrent neural network architecture, a two-layer feedforward neural network architecture, a three-layer feedforward neural network architecture, and/or the like) to perform pattern recognition with regard to patterns in the historical information. In this case, use of the artificial neural network processing technique may improve an accuracy of a supervised learning model generated by the processing device by being more robust to noisy, imprecise, or incomplete data, and by enabling the processing device to detect patterns and/or trends undetectable to human analysts or systems using less complex techniques. Furthermore, when using a recurrent neural network architecture, long short-term memory (LSTM) may be employed to classify, make predictions, and/or otherwise process time-series data, which may be useful to predict how patterns change over time (e.g., over a month, a year, and/or the like).

In this way, the processing device may use artificial intelligence techniques, machine learning techniques, deep learning techniques, and/or the like to determine one or more associations between historical information and a determination indicating a type of tissue.

In some implementations, the processing device may obtain the machine learning model from a different device that generates and/or trains the machine learning model (e.g., in a similar manner as described herein). For example, the different device may send the machine learning model to the processing device and/or the user device may request and receive the machine learning model from the different device. In some implementations, the different device may update and send (e.g., on a scheduled basis, on an on-demand basis, on a triggered basis, and/or the like) the machine learning model to the processing device and/or the processing device may request and receive the updated machine learning model from the different device.

As shown by reference number 418, the processing device may cause the display screen to display information concerning the ultrasound data and/or the type of tissue at the face of the drill bit as well as a location of the brill bit tip. For example, the processing device may cause the display screen to display information concerning a current drilling trajectory of the drill bit and information concerning the type of tissue at the face of the drill bit. The processing device may cause display of a first indicator to indicate that the operator of the surgical device is to stop and adjust a current drilling trajectory (e.g., to avoid directly damaging a critical tissue, such as a nerve), a second indicator to indicate that the operator is maintain the current drilling trajectory (e.g., to maintain drilling of a target tissue, such as cancellous bone), a third indicator to indicate that the operator is to be cautious about maintaining the current drilling trajectory (e.g., to avoid accidentally damaging a tissue, such as cortical bone), and/or the like. As another example, the processing device may cause the display screen to display information concerning a current drilling trajectory of the drill bit onto a pre-obtained image of a targeted tissue (e.g., a computed tomography (CT) scan image, an X-Ray image, and/or the like of a vertebra). In addition, a priori data (such as temporal data from a previous recording, or data from an electromagnetic tracker, or tracking data from a robot arm attached to the surgical device) may be used to filter out the signal associated with the drill bit tip.

Additionally, or alternatively, the processing device may generate, based on the type of tissue at the face of drill bit, at least one instruction regarding operation of the drill bit. For example, the processing platform may generate at least one instruction to cease operating the drill bit when the processing device determines that the tissue at the face of the drill bit is a blood vessel. The processing device may cause the display screen to display a message based on the at least one instruction. Additionally, or alternatively, the processing device may send the at least one instruction to the surgical device to cause the drill to operate based on the at least one instruction (e.g., the surgical device may execute the at least one instruction to cause the drill bit to start or stop rotating). In this way, the processing device may be able to automatically control operation of the surgical device and/or the drill bit to ensure that targeted tissue is drilled and/or that collateral tissue is not damaged.

As indicated above, FIGS. 4A-4C are provided merely as examples. Other examples may differ from what is described with regard to FIGS. 4A-4C. Further, the number and arrangement of devices and networks shown in FIGS. 4A-4C are provided as an example. In practice, there may be additional devices and/or networks, fewer devices and/or networks, different devices and/or networks, or differently arranged devices and/or networks than those shown in FIGS. 4A-4C. Furthermore, two or more devices shown in FIGS. 4A-4C may be implemented within a single device, or a single device shown in FIGS. 4A-4C may be implemented as multiple, distributed devices. Additionally, or alternatively, a set of devices (e.g., one or more devices) of example implementation 100 may perform one or more functions described as being performed by another set of devices of example implementation 100.

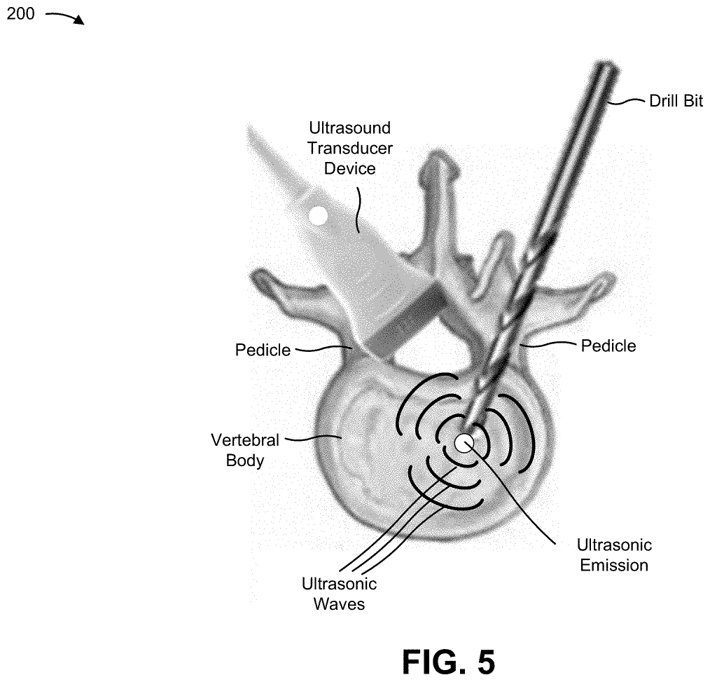

FIG. 5 is a diagram of an example surgical procedure using the devices described herein. In this example, an operator of the surgical device is conducting a spinal fusion surgery and may need to drill holes in the pedicles of a vertebra (e.g., to place a screw in the pedicle that connects to a metal rod). A pedicle exists on either side of the vertebra adjacent to the spinal cord. An outer region of the pedicle consists of cortical bone that surrounds a more porous, cancellous bone core. A preferred place of a screw in the pedicle is the cancellous core of the pedicle.

As shown in FIG. 5, the operator may operate the surgical device to drill a hole in the pedicle. The drill bit may rotate (e.g., spin on an axis) to remove bone from the pedicle to create the hole. In some implementations, as described herein, the operator may cause the laser light source to generate laser light that propagates to a face of the drill bit, which may create an ultrasonic emission comprising one or more ultrasonic waves at the face of the drill bit. The drill bit may be stationary while the laser light creates the ultrasonic emission or the drill bit may be rotating. In either case, the laser light may propagate through an internal optical fiber of the drill bit to the face of the drill bit.

As further shown in FIG. 5, the operator may place the ultrasound transducer in a position to detect the ultrasonic emission. The ultrasound transducer may obtain ultrasound data associated with the ultrasonic emission and may send the ultrasound data to the processing device for processing, as described herein. In this way, the operator may learn information (e.g., information about a drill path trajectory of the drill bit, information about the location of the drill bit tip, information concerning whether the drill bit is drilling cortical bone or cancellous bone, and/or the like) that may facilitate the operator accurately drilling the hole in the pedicle. For example, the operator may use the information to determine a starting point and trajectory for drilling the hole in the pedicle, which may reduce a likelihood that the drill bit will disrupt or breach the cortical bone that surrounds the cancellous bone core of the pedicle.

As indicated above, FIG. 5 is provided merely as an example. Other examples may differ from what is described with regard to FIG. 5.

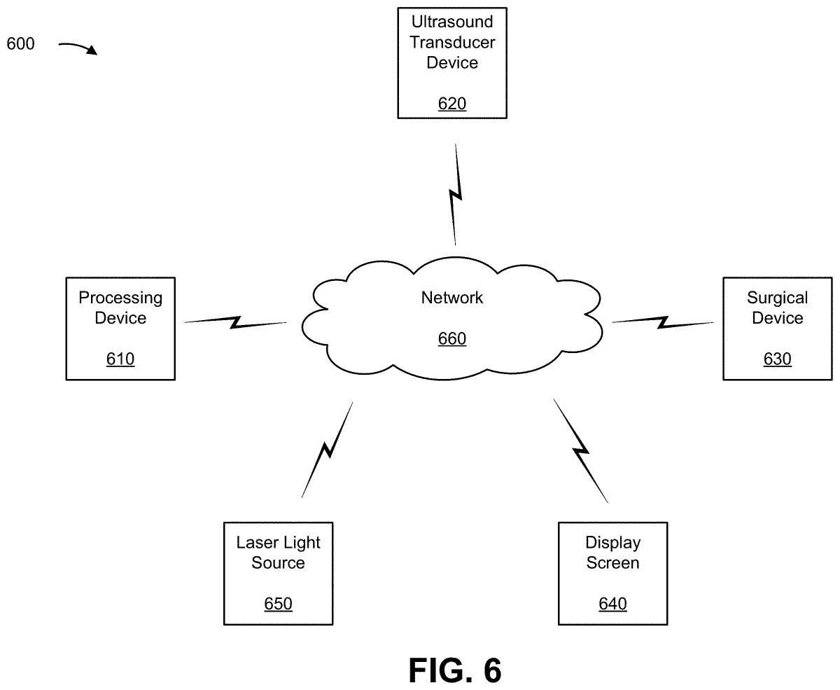

FIG. 6 is a diagram of an example environment 600 in which systems and/or methods described herein may be implemented. As shown in FIG. 6, environment 600 may include a processing device 610, an ultrasound transducer device 620, a surgical device 630, a display screen 640, a laser light source 650, a network 660, and/or the like. Devices of environment 600 may interconnect via wired connections, wireless connections, or a combination of wired and wireless connections.

Processing device 610 includes one or more devices capable of receiving, generating, storing, processing, and/or providing information, such as information described herein. For example, processing device 610 may include a computer (e.g., a desktop computer, a laptop computer, a tablet computer, a handheld computer, a server device, and/or the like), a mobile phone (e.g., a smart phone, a radiotelephone, and/or the like), or a similar device. In some implementations, the processing device may cause laser light source 650 to generate laser light, may obtain ultrasound data from ultrasound transducer device 620, may process the ultrasound data, and may cause at least one action to be performed based on processing the ultrasound data. In some implementations, processing device 610 may communicate (e.g., via network 660) with ultrasound transducer device 620, surgical device 630, display screen 640, and/or laser light source 650.

Ultrasound transducer device 620 includes one or more devices capable of receiving, generating, storing, processing, and/or providing information, such as information described herein. For example, ultrasound transducer device 620 may include one or more ultrasound transducers to obtain ultrasound data. In some implementations, ultrasound transducer device 620 may obtain ultrasound data associated with ultrasonic emissions created by laser light stimulating biological tissue. In some implementations, ultrasound transducer device 620 may communicate (e.g., via network 660) with processing device 610, surgical device 630, and/or display screen 640.

Surgical device 630 includes one or more devices capable of receiving, generating, storing, processing, and/or providing information, such as information described herein. Surgical device 630 may include a battery, a motor, an optical fiber, an optical interface, one or more gears, a drill bit, a structure to hold the drill bit, an operating switch, and/or the like. In some implementations, surgical device 630 may include processing device 610 and/or laser light source 650 within a housing of surgical device 630. In some implementations, display screen 640 may be attached to surgical device 630 (e.g., to the housing of surgical device 630). In some implementations, surgical device 630 may communicate (e.g., via network 660) with processing device 610, ultrasound transducer device 620, display screen 640, and/or laser light source 650.

Display screen 640 includes one or more devices capable of receiving, generating, storing, processing, and/or providing information, such as information described herein. Display screen 640 may include any digital or analog display that is capable of presenting audio and/or video content. Display screen 640 may include technologies, such as cathode ray tube (CRT) displays, liquid crystal displays (LCDs), light-emitting diode (LED) displays, plasma displays, and/or the like. Examples of display screen 640 may include a television, a projector, a computer monitor, and/or other types of devices capable of presenting audio and/or video content. In some implementations, surgical device 630 may communicate (e.g., via network 660) with processing device 610, ultrasound transducer device 620, and/or surgical device 630.

Laser light source 650 includes one or more devices capable of receiving, generating, storing, processing, and/or providing information, such as information described herein. Laser light source 650 may include a laser diode, such as a pulsed laser diode, or another laser light emitting device for generating laser light. Laser light source 650 may be configured to produce laser light with a particular wavelength, energy, fluence, and/or the like. In some implementations, laser light source 650 may communicate (e.g., via network 660) with processing device 610 and/or surgical device 630.

Network 660 includes one or more wired and/or wireless networks. For example, network 660 may include a cellular network (e.g., a long-term evolution (LTE) network, a code division multiple access (CDMA) network, a 3G network, a 4G network, a 5G network (e.g., a 5G mmW network), another type of next generation network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the internet, a fiber optic-based network, a cloud computing network, a mesh network and/or the like, and/or a combination of these or other types of networks.

The number and arrangement of devices and networks shown in FIG. 6 are provided as one or more examples. In practice, there may be additional devices and/or networks, fewer devices and/or networks, different devices and/or networks, or differently arranged devices and/or networks than those shown in FIG. 6. Furthermore, two or more devices shown in FIG. 6 may be implemented within a single device, or a single device shown in FIG. 6 may be implemented as multiple, distributed devices. Additionally, or alternatively, a set of devices (e.g., one or more devices) of environment 600 may perform one or more functions described as being performed by another set of devices of environment 600.

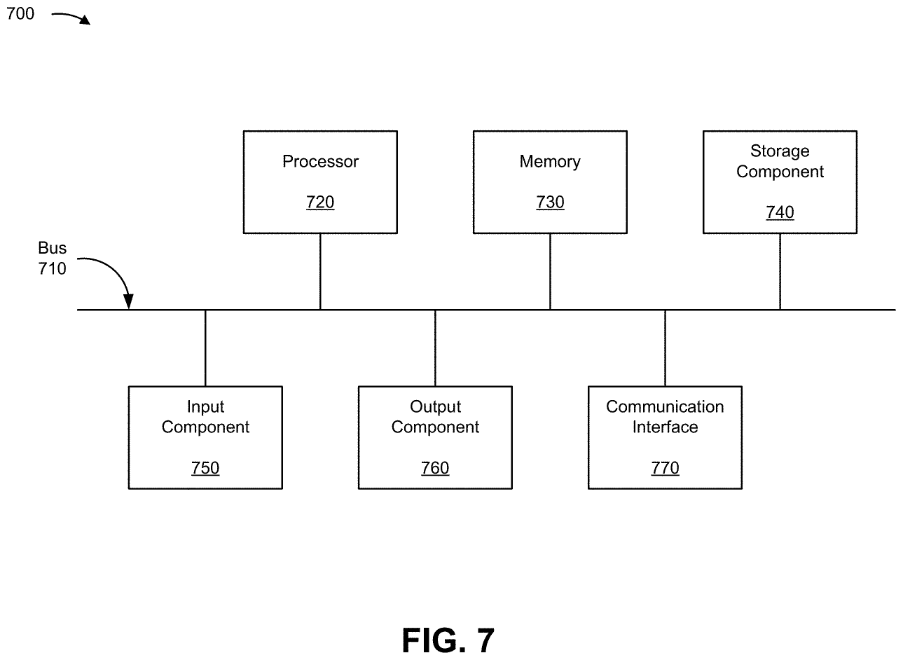

FIG. 7 is a diagram of example components of a device 700. Device 700 may correspond to processing device 610, ultrasound transducer device 620, surgical device 630, display screen 640, and/or laser light source 650. In some implementations processing device 610, ultrasound transducer device 620, surgical device 630, display screen 640, and/or laser light source 650 may include one or more devices 700 and/or one or more components of device 700. As shown in FIG. 7, device 700 may include a bus 710, a processor 720, a memory 730, a storage component 740, an input component 750, an output component 760, and a communication interface 770.

Bus 710 includes a component that permits communication among multiple components of device 700. Processor 720 is implemented in hardware, firmware, and/or a combination of hardware and software. Processor 720 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 720 includes one or more processors capable of being programmed to perform a function. Memory 730 includes a random access memory (RAM), a read only memory (ROM), and/or another type of dynamic or static storage device (e.g., a flash memory, a magnetic memory, and/or an optical memory) that stores information and/or instructions for use by processor 720.

Storage component 740 stores information and/or software related to the operation and use of device 700. For example, storage component 740 may include a hard disk (e.g., a magnetic disk, an optical disk, and/or a magneto-optic disk), a solid state drive (SSD), a compact disc (CD), a digital versatile disc (DVD), a floppy disk, a cartridge, a magnetic tape, and/or another type of non-transitory computer-readable medium, along with a corresponding drive.

Input component 750 includes a component that permits device 700 to receive information, such as via user input (e.g., a touch screen display, a keyboard, a keypad, a mouse, a button, a switch, and/or a microphone). Additionally, or alternatively, input component 750 may include a component for determining location (e.g., a global positioning system (GPS) component) and/or a sensor (e.g., an accelerometer, a gyroscope, an actuator, another type of positional or environmental sensor, and/or the like). Output component 760 includes a component that provides output information from device 700 (via, e.g., a display, a speaker, a haptic feedback component, an audio or visual indicator, and/or the like).

Communication interface 770 includes a transceiver-like component (e.g., a transceiver, a separate receiver, a separate transmitter, and/or the like) that enables device 700 to communicate with other devices, such as via a wired connection, a wireless connection, or a combination of wired and wireless connections. Communication interface 770 may permit device 700 to receive information from another device and/or provide information to another device. For example, communication interface 770 may include an Ethernet interface, an optical interface, a coaxial interface, an infrared interface, a radio frequency (RF) interface, a universal serial bus (USB) interface, a Wi-Fi interface, a cellular network interface, and/or the like.

Device 700 may perform one or more processes described herein. Device 700 may perform these processes based on processor 720 executing software instructions stored by a non-transitory computer-readable medium, such as memory 730 and/or storage component 740. As used herein, the term “computer-readable medium” refers to a non-transitory memory device. A memory device includes memory space within a single physical storage device or memory space spread across multiple physical storage devices.

Software instructions may be read into memory 730 and/or storage component 740 from another computer-readable medium or from another device via communication interface 770. When executed, software instructions stored in memory 730 and/or storage component 740 may cause processor 720 to perform one or more processes described herein. Additionally, or alternatively, hardware circuitry may be used in place of or in combination with software instructions to perform one or more processes described herein. Thus, implementations described herein are not limited to any specific combination of hardware circuitry and software.

The number and arrangement of components shown in FIG. 7 are provided as an example. In practice, device 700 may include additional components, fewer components, different components, or differently arranged components than those shown in FIG. 7. Additionally, or alternatively, a set of components (e.g., one or more components) of device 700 may perform one or more functions described as being performed by another set of components of device 700.

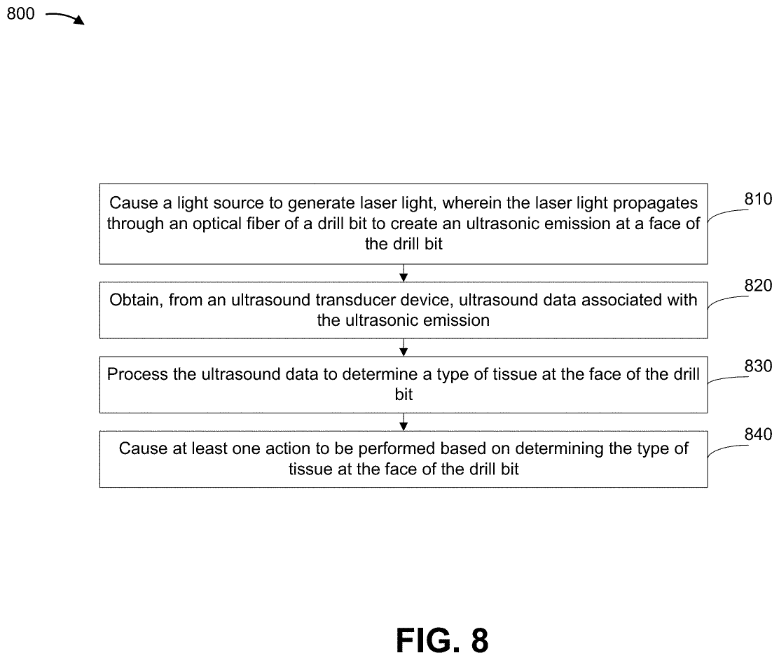

FIG. 8 is a flowchart of an example process 800 for internal laser light delivery for photoacoustic-guided drilling. In some implementations, one or more process blocks of FIG. 8 may be performed by a processing device (e.g., processing device 610). In some implementations, one or more process blocks of FIG. 8 may be performed by another device or a group of devices separate from or including the processing device, such as an ultrasound transducer device (e.g., ultrasound transducer device 620), a surgical device (e.g., surgical device 630), a display screen (e.g., display screen 640), a laser light source (e.g., laser light source 650), and/or the like.

As shown in FIG. 8, process 800 may include causing a light source to generate laser light, wherein the laser light propagates through an optical fiber of a drill bit to create an ultrasonic emission at a face of the drill bit (block 810). For example, the processing device (e.g., using processor 720, memory 730, storage component 740, input component 750, output component 760, communication interface 770, and/or the like) may cause, by a device, a light source to generate laser light, as described above. In some implementations, the laser light propagates through an optical fiber of a drill bit to create an ultrasonic emission at a face of the drill bit.

As further shown in FIG. 8, process 800 may include obtaining, from an ultrasound transducer device, ultrasound data associated with the ultrasonic emission (block 820). For example, the processing device (e.g., using processor 720, memory 730, storage component 740, input component 750, output component 760, communication interface 770, and/or the like) may obtain, from an ultrasound transducer device, ultrasound data associated with the ultrasonic emission, as described above.

As further shown in FIG. 8, process 800 may include processing the ultrasound data to determine a type of tissue at the face of the drill bit (block 830). For example, the processing device (e.g., using processor 720, memory 730, storage component 740, input component 750, output component 760, communication interface 770, and/or the like) may process the ultrasound data to determine a type of tissue at the face of the drill bit, as described above.

As further shown in FIG. 8, process 800 may include causing at least one action to be performed based on determining the type of tissue at the face of the drill bit (block 840). For example, the processing device (e.g., using processor 720, memory 730, storage component 740, input component 750, output component 760, communication interface 770, and/or the like) may cause at least one action to be performed based on determining the type of tissue at the face of the drill bit, as described above.

Process 800 may include additional implementations, such as any single implementation or any combination of implementations described below and/or in connection with one or more other processes described elsewhere herein.

In some implementations, causing the light source to generate the laser light comprises: obtaining input regarding a user preference regarding an energy, a fluence, or a wavelength of the laser light; and causing the light source to generate the laser light based on the user preference.

In some implementations, processing the ultrasound data to determine the type of tissue at the face of the drill bit comprises processing the ultrasound data using a machine learning model to determine the type of tissue at the face of the drill bit.

In some implementations, causing the at least one action to be performed comprises causing a screen display to display information concerning the ultrasound data and the type of tissue at the face of the drill bit.

In some implementations, causing the at least one action to be performed comprises: generating, based on the type of tissue at the face of the drill bit, at least one instruction regarding operation of the drill bit; and causing the drill bit to operate based on the at least one instruction.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

The foregoing disclosure provides illustration and description, but is not intended to be exhaustive or to limit the implementations to the precise forms disclosed. Modifications and variations may be made in light of the above disclosure or may be acquired from practice of the implementations.

As used herein, the term “component” is intended to be broadly construed as hardware, firmware, and/or a combination of hardware and software.

Some implementations are described herein in connection with thresholds. As used herein, satisfying a threshold may, depending on the context, refer to a value being greater than the threshold, more than the threshold, higher than the threshold, greater than or equal to the threshold, less than the threshold, fewer than the threshold, lower than the threshold, less than or equal to the threshold, equal to the threshold, or the like.

Certain user interfaces have been described herein and/or shown in the figures. A user interface may include a graphical user interface, a non-graphical user interface, a text-based user interface, and/or the like. A user interface may provide information for display. In some implementations, a user may interact with the information, such as by providing input via an input component of a device that provides the user interface for display. In some implementations, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

It will be apparent that systems and/or methods described herein may be implemented in different forms of hardware, firmware, or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be designed to implement the systems and/or methods based on the description herein.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A device, comprising:
a first optical fiber; and
an optical interface connected to the first optical fiber,
wherein the optical interface is configured to transfer laser light from the first optical fiber to a second optical fiber in a drill bit.

2. The device of claim 1, wherein the second optical fiber propagates the laser light to create an ultrasonic emission at a face of the drill bit.

3. The device of claim 1, wherein the device includes a laser light source that generates the laser light, and
wherein the laser light source is a pulsed laser diode.

4. The device of claim 1, wherein the device includes a laser light source that generates the laser light, the first optical fiber, and the optical interface within a housing of the device.

5. The device of claim 1, wherein an end of the first optical fiber is inserted into a first end of the optical interface and a shank of the drill bit is to be inserted into a second end of the optical interface,
wherein the optical interface is to align the end of the first optical fiber and the shank of the drill bit to allow the laser light to transfer from the first optical fiber to the second optical fiber in the drill bit.

6. The device of claim 5, wherein the end of the first optical fiber and the shank of the drill bit are to be separated by a distance that satisfies a threshold.

7. The device of claim 1, further comprising:
the drill bit with the second optical fiber,
wherein the second optical fiber propagates the laser light to create an ultrasonic emission at a face of the drill bit.

8. The device of claim 7, further comprising:
one or more ultrasound transducers to obtain ultrasound data concerning the ultrasonic emission;
one or more processing components to process the ultrasound data to determine a type of tissue at the face of the drill bit; and
a screen display to display information concerning the type of tissue at the face of the drill bit.

9. A shaft, comprising:
a face of the shaft that is configured to penetrate bone; and
an optical fiber within the shaft to propagate laser light generated by a laser light source to the face of the shaft to create an ultrasonic emission at the face of the shaft.

10. The shaft of claim 9, wherein the shaft has one or more holes in the face of the shaft to facilitate propagating the laser light to the face of the shaft.

11. The shaft of claim 10, wherein the one or more holes are implanted with diamond lenses.

12. The shaft of claim 9, wherein the shaft is composed of a material to absorb a wavelength of light associated with the laser light.

13. The shaft of claim 9, wherein the shaft is a drill bit, a needle, or a pedicle probe.

14. The shaft of claim 9, wherein the shaft is to create a hole in a bone using one or more cutting edges.

15. The shaft of claim 9, wherein an end of the shaft is to be inserted into an end of an optical interface of a surgical drill device,
wherein the optical interface transfers the laser light generated by the laser light source to the optical fiber.

16. A method, comprising:
causing, by a device, a light source to generate laser light,
wherein the laser light propagates through an optical fiber of a drill bit to create an ultrasonic emission at a face of the drill bit;
obtaining, by the device and from an ultrasound transducer device, ultrasound data associated with the ultrasonic emission;
processing, by the device, the ultrasound data to determine a location of the drill bit tip;
processing, by the device, the ultrasound data to determine a type of tissue at the face of the drill bit; and
causing, by the device, at least one action to be performed based on determining the type of tissue at the face of the drill bit.

17. The method of claim 16, wherein causing the light source to generate the laser light comprises:
obtaining input regarding a user preference regarding an energy, a fluence, or a wavelength of the laser light; and
causing the light source to generate the laser light based on the user preference.

18. The method of claim 16, wherein processing the ultrasound data to determine the type of tissue at the face of the drill bit comprises:
processing the ultrasound data using a machine learning model to determine the type of tissue at the face of the drill bit.

19. The method of claim 16, wherein causing the at least one action to be performed comprises:
causing a screen display to display information concerning the ultrasound data and the type of tissue at the face of the drill bit.

20. The method of claim 16, wherein causing the at least one action to be performed comprises:
generating, based on the type of tissue at the face of the drill bit, at least one instruction regarding operation of the drill bit; and
causing the drill bit to operate based on the at least one instruction.
